(19) United States
(12) Patent Application Publication
(10) Pub. No.: US 2008/0167639 A1
(43) Pub. Date: Jul. 10, 2008

(54) Methods for localized intra-body treatment of tissue

(75) Inventor: Pinhas Gilboa, Haifa (IL)

Correspondence Address: INSKEEP INTELLECTUAL PROPERTY GROUP, INC, 2281 W. 190TH STREET, SUITE 200, TORRANCE, CA, 90504, US

(73) Assignee: superDimension Ltd.

(21) Appl. No.: 11/571,796

(22) Filed: Jan. 8, 2007

(51) Int. Cl. A61M 5/00

(52) U.S. Cl. 604/514

(57) ABSTRACT

A method for local treatment of a target region of tissue within a body of a subject includes navigating a first probe (210, 310) intra-bodily to a first location within the body of the subject and employing the first probe to locally change at least one characteristic of a selected region of tissue. Either the same probe (210, 310) or another probe (250, 330) is employed to apply a treatment to the target region of the body of the subject. The changed characteristic of the selected region of tissue is chosen so as to enhance at least one parameter of the treatment.

### RELATED APPLICATIONS

This application claims priority to U.S. Provisional Application Ser. No. 60/585,570 filed Jul. 7, 2004 entitled Method For Ablating Tissue and International Patent Application PCT/IL2005/000724, International Filing Date 7 Jul. 2005, entitled Methods For Localized Intra-Body Treatment Of Tissue, both of which are hereby incorporated by reference.

### BACKGROUND OF THE INVENTION

The present invention relates to methods for localized intra-body treatment of tissue and, in particular, it concerns intra-body methods of treatment wherein two actions on the same tissue or adjacent regions of tissue provide various synergous effects.

This invention relates to PCT application WO 03/086498 titled ‘Endoscope Structure and Techniques for Navigation in Brunched Structure' to Gilboa, fully incorporated herein by reference. Said patent application describes a method and apparatus in which a thin locatable guide, enveloped by a sheath, is used to navigate a bronchoscopic tool to a target location within the lung, aimed in particular to deliver treatments to the lung periphery beyond the bronchoscope's own reach. The coordinates of the target are predetermined based upon three-dimensional CT data. A location sensor is incorporated at the locatable guide's tip. The enveloped guide is inserted into the lung via the working channel of a bronchoscope. First, the bronchoscope's tip is directed to the furthest reachable location in the direction of the target. Next, the guide is advanced beyond the tip of the bronchoscope towards the designated target, based on the combination of the CT data and the position of the guide's tip as measured in body coordinates. When the guide's tip is at the target, the guide is withdrawn, freeing the sheath for insertion of a bronchoscopic tool. In order to prevent the distal end portion of the sheath from sliding away from the target, the sheath is locked to the bronchoscope's body and the bronchoscope itself is held steadily to prevent it from slipping further into the lungs or outwards. Because the airways in the periphery of the lung are narrow, approximately in the same dimensions as the sheath, sideways movements are extremely limited. The above system and apparatus are aimed to navigate standard bronchoscopic tools to a target located in the lung. In its basic operation, first the target is identified in the CT data, then the guide is navigated to the target and a medical treatment is delivered. It would be advantageous, however, to perform more sophisticated treatments, such as by combining different types of treatments into a single session.

### OBJECTS AND SUMMARY OF THE INVENTION

The methods of the present invention allow performance of intra-body treatments to tissue by combining different types of actions or treatments in a single session. The purpose is to enhance the overall efficacy more than that achieved by each separately. This is achieved if the effect of each of the treatments is amplified by the effect of the other. Because the airways in the periphery of the lung are very narrow, having a diameter as low as less than 1 mm, it is often impossible to bring more than one catheter to target concurrently. Hence, either the two (or more) treatments will be performed by the same probe, or different probes, each for a single treatment, will be guided sequentially to the target and operated to deliver each its own treatment. Navigability or, more precisely, the capability of precise endoscopic localization is essential in such catheter-based treatment in the lung and elsewhere.

Thus, according to the teachings of the present invention there is provided, a method for local treatment of a target region of tissue within a body of a subject, the method comprising: (a) navigating a first probe intra-bodily to a first location within the body of the subject; (b) employing the first probe to locally change at least one characteristic of a selected region of tissue, the selected region of tissue being in known spatial relation to the target region of tissue; and (c) employing a treatment probe selected from the group consisting of the first probe and a second probe to apply a treatment to the target region of the body of the subject, wherein the changed characteristic of the selected region of tissue is chosen so as to enhance at least one parameter of the treatment.

It should be noted that the “navigating” of the probe(s) to the required positions may be achieved either by using directly steerable probes, or according to the technique of the above-referenced PCT publication wherein the probe(s) need not themselves be steerable and are guided by a sleeve, as will be illustrated below.

It should also be noted that the “characteristic” of the tissue may be any characteristic which is varied in either a permanent or temporary manner. Various non-limiting examples will be given below. Similarly, the “treatment” may be any type of treatment in the broadest sense of the term. Here too, various non-limiting examples re given below. The at least one “parameter of the treatment” enhanced by the change in characteristic may be any parameter related to the treatment including, but not limited to: efficacy of the treatment, efficiency of the treatment, localization or targeting of the effect of the treatment, or reduction of a side effect of the treatment.

According to a further feature of the present invention, navigation of the first probe includes measuring a location of the probe relative to the body of the subject.

According to a further feature of the present invention, the measuring is performed by determining a position of a location sensor associated with the first probe, the location sensor being part of an electromagnetic tracking system.

According to a further feature of the present invention, the navigation further includes determining a location of the first probe within an image of at least the target region of tissue, the image being derived from a medical imaging system.

According to a further feature of the present invention, the medical imaging system is selected from the group consisting of: computer tomography, magnetic resonance, nuclear camera, PET and ultrasound imaging.

According to a further feature of the present invention, the treatment probe is the first probe.

According to a further feature of the present invention, the first probe includes a first portion configured for changing the characteristic of the selected region of tissue and a second portion configured for applying the treatment.

According to a further feature of the present invention, the characteristic is a physical characteristic of the tissue.

According to a further feature of the present invention, the physical characteristic is the heat capacity of the tissue.

According to a further feature of the present invention, the physical characteristic is the heat dissipation from the tissue.

According to a further feature of the present invention, the physical characteristic is electrical conductivity of the tissue.

According to a further feature of the present invention, the characteristic is a physiological characteristic of the tissue.

According to a further feature of the present invention, the physiological characteristic is the vitality of the tissue.

According to a further feature of the present invention, the physiological characteristic is the rate of blood flow in the tissue.

According to a further feature of the present invention, the characteristic is a biological characteristic of the tissue.

According to a further feature of the present invention, the biological characteristic is changed by marking individual cells.

According to a further feature of the present invention, the marking the cells is performed by implant of receptors.

According to a further feature of the present invention, the biological characteristic is changed by increasing the sensitivity of the tissue to the action of a specific material.

According to a further feature of the present invention, at least one of the first probe and the treatment probe is a needle for injection.

According to a further feature of the present invention, at least one of the first probe and the treatment probe is a sprayer for spaying a substance to be absorbed into the tissue.

According to a further feature of the present invention, the first probe includes a stent coated with a drug.

According to a further feature of the present invention, at least one of the first probe and the treatment probe is a drug delivery device.

According to a further feature of the present invention, the at least one characteristic of the tissue is changed by application of bio-chemicals into the tissue.

According to a further feature of the present invention, the at least one characteristic of the tissue is changed by application of chemicals into the tissue.

According to a further feature of the present invention, the at least one characteristic of the tissue is changed by application of biological materials into the tissue.

According to a further feature of the present invention, the at least one characteristic of the tissue is changed by application of micro or nano particles into the tissue.

According to a further feature of the present invention, the at least one characteristic of the tissue is changed by application of glue into the tissue.

According to a further feature of the present invention, the at least one characteristic of the tissue is changed by application of micro coils into the tissue.

According to a further feature of the present invention, the treatment includes transferring energy between the treatment probe and the tissue of the target region.

According to a further feature of the present invention, the treatment is an ablation of tissue in at least part of the target region and wherein the treatment probe is an ablation device.

According to a further feature of the present invention, the transferred energy is radiofrequency ablation energy and the treatment probe is an electrode for delivering the radiofrequency ablation energy.

According to a further feature of the present invention, the treatment probe is a heat absorber for freezing the tissue.

According to a further feature of the present invention, the treatment probe includes a radioactive emitter and the transferred energy is radioactive radiation.

According to a further feature of the present invention, the radioactive radiation is gamma radiation.

According to a further feature of the present invention, the energy is selected from the group consisting of: electrical energy, coherent and noncoherent electromagnetic waves including infrared, visible light, ultra violet, radio frequency, microwaves and gamma radiation, pressure, pressure waves, mechanical shocks, sonic, ultrasonic, emission of particles and thermal energy.

According to a further feature of the present invention, the changing is effective to increase an energy coupling between the treatment probe and the tissue of the target region.

According to a further feature of the present invention, the changing is effective to increase an energy insulation between the tissue at the target region and at least one adjacent region of tissue.

According to a further feature of the present invention, the changing is effective to increase a number of dead cells in the tissue at the target region.

According to a further feature of the present invention, the treatment is applied while the treatment probe is positioned substantially at the first location.

According to a further feature of the present invention, the treatment is applied while the treatment probe is positioned at a second location displaced relative to the first location.

According to a further feature of the present invention, the treatment includes application of bio-chemicals into the tissue.

According to a further feature of the present invention, the treatment includes application of chemicals into the tissue.

According to a further feature of the present invention, the treatment is chemotherapy.

According to a further feature of the present invention, the treatment includes application of biological materials into the tissue.

According to a further feature of the present invention, the treatment includes application of micro or nano particles into the tissue.

According to a further feature of the present invention, the treatment includes application of glue into the tissue.

According to a further feature of the present invention, the treatment includes application of micro coils into the tissue.

According to a further feature of the present invention, the changing at least one characteristic of the tissue is effected from ablation of the tissue.

According to a further feature of the present invention, the ablation of tissue is performed by chemotherapy techniques.

According to a further feature of the present invention, the ablation of tissue is performed by an energy method of ablation.

According to a further feature of the present invention, the treatment includes application of a drug at the target region.

According to a further feature of the present invention, the drug is selected from the group consisting of: antibiotics, pain relief, anti-inflammatory, gene therapy, enzymatic drug, bleeding inhibitory and chemo-agents.

According to a further feature of the present invention, the changing the at least one characteristic of the tissue and the treatment are performed concurrently.

According to a further feature of the present invention, the changing the at least one characteristic of the tissue is performed after the treatment.

According to a further feature of the present invention, the navigating is performed using at least one endoscopic localization technique selected from the group consisting of: electromagnetic location sensing; magnetic location sensing; image correlation; and trigonometry based upon at least one non-invasive imaging device.

According to a further feature of the present invention, the selected region of tissue is substantially coincident with the target region of tissue.

According to a further feature of the present invention, the selected region of tissue is substantially adjacent to the target region of tissue.

### BRIEF DESCRIPTION OF THE DRAWINGS

The invention is herein described, by way of example only, with reference to the accompanying drawings, wherein:

FIG. 1 is a schematic isometric representation of an apparatus as described in PCT patent application publication no. WO 03/086498 for use in implementing the method of the present invention;

FIG. 2a is a schematic illustration of a sheath positioned adjacent to a target region of tissue according to the teachings of the present invention;

FIG. 2b is a schematic view similar to FIG. 2a illustrating use of a first probe to locally change at least one characteristic of a selected region of tissue according an implementation of the present invention;

FIG. 2c is a schematic view similar to FIG. 2a illustrating use of a treatment probe to apply a treatment to a target region of the body of the subject according an implementation of the present invention; and

FIGS. 3a-3d are schematic isometric cut-away views illustrating the steps of a method according to a second implementation of the present invention for delivering energy to a target region of tissue.

### DETAILED DESCRIPTION OF THE INVENTION

The present invention is a method for local treatment of a target region of tissue within a body of a subject.

The principles and operation of methods according to the present invention may be better understood with reference to the drawings and the accompanying description.

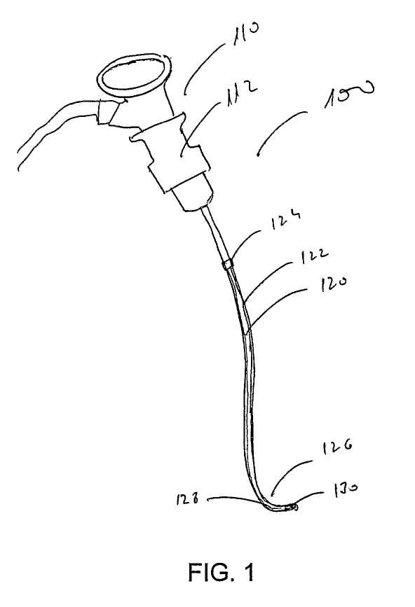

Referring now to the drawings, FIG. 1 describes the basic method of navigation according to PCT application WO 03/086498. There is shown a locatable guide 100, having proximal end 110 and a distal end 126. The distal end comprises a deflection mechanism 128 and a location sensor 130. The proximal end comprises a steering lever by which the practitioner can deflect

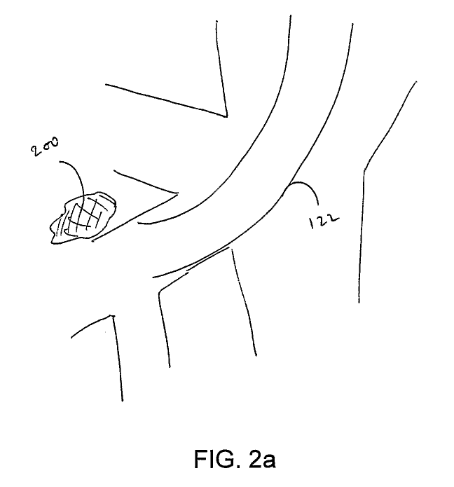

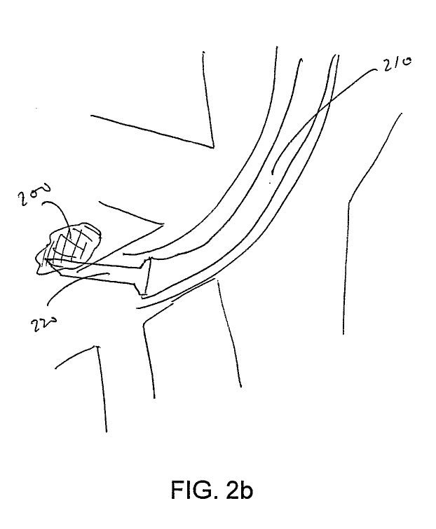

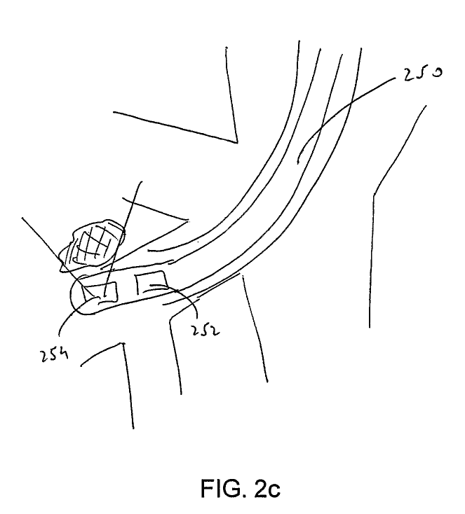

the distal end portion in any desired direction. The shaft 120 of the locatable guide is enveloped by a sheath 122. When the distal tip of the guide is on target 200, the sheath is disconnected from the guide by opening lock 124, pulling the guide out and leaving the sheath empty in the pulmonary airway as shown in FIG. 2a. The location of the location sensor is tracked by an electromagnetic tracking system (not shown). More details are explained in WO 03/086498, as well as methods of navigation by measuring the coordinates of the location sensor relative to the tissue and also relative to the image made by a medical imaging device such as CT MRI, nuclear camera, PET and ultrasound.

FIGS. 2b and 2c show the fundamentals of an exemplary embodiment of this patent. In the first phase, a treatment is applied to change the characteristic of the tissue at a target in the lung as preparation for the next treatment. As shown in FIG. 2b, it is preferably done by first navigating the locatable guide to the target, than freeing the sheath by withdrawing the guide and leaving the sheath empty, and than inserting catheter 210 up to target 200. In the example shown, the treatment is an injection of substance into target 200 using an injection needle 220 that is mounted at the proximal end of catheter 210. Most likely, the target is located behind the airway wall, at the side of the tip of catheter 210. Inserting the needle to the right location in the target needs to be performed in an accurate angle in addition to the necessity for being in the precise location. This can be achieved by various techniques, such as the use of a suitable steering mechanism in combination with a 6-degrees-of-freedom position sensor.

In phase two, a second catheter 250 is inserted. In the example shown in FIG. 2c the probe is a probe for inducing energy via an energy transmitter 254. The location of the tip of the second probe does not necessarily coincide with the location of the tip of the first probe. In fact, for many combined treatments, it is necessary to change the tip's location between the first and the second treatment.

Preferably, a location sensor is implemented in each probe, such as sensor 252 in the above example. However, because of the limited space available, and because of cost considerations, in many applications implementing a location sensor into the catheter's tip is not feasible. In such instances, the navigation of each probe will be performed according to the methods describe in WO 03/086498 by using the locatable guide and sheath as explained above. Those probes may also incorporate a steering mechanism similar to that of the locatable guide.

It should be noted that, although a preferred implementation is described herein with reference to a location sensor of a type described in the aforementioned PCT publication and citations therein, the broad scope of the present invention includes cases employing all known techniques for endoscopic localization which are able to establish precisely the position of the treatment probes within the body. Other examples include, but are not limited to: image registration based on endoscopic images compared to virtual endoscopy; and trigonometric localization based on plural fluoroscopic images taken at different angles.

It is sometimes the case that both probes need to be combined into a single probe. That is either because it is actually the same device, as in the case where the treatment uses the same technique (for example is the injection of two complementary substances to the same location or injecting into two different locations of the same substance), or because there is a reason to perform both treatments concurrently (as in the case where micro or nano particles are injected into a lesion and energy is exerted onto the lesion, activating this particles killing the cancerous cells).

The reason to change some characteristic of the tissue before applying the second treatment is to overcome some limitations that limit or reduce the effectiveness of the second treatment. Ablating a tissue by heating or cooling is one example. The effectiveness of the treatment depends of the energy couple between the probe and the tissue, and the power dissipation rate from that tissue.

Analysis of the effectiveness of radiofrequency (RF) ablation is brought here as an example. RF ablation is performed by inducing alternating electric current through the tissue. The current flows from an electrode into the tissue. The first parameter is the conductivity between the electrode and the tissue. Since the air that surrounds the probe in the airway is insulated, there might be a need to fill the gap between the electrode and the airway wall with a high conducting gel. In that case, applying the gel is the first treatment and the ablation is the second treatment.

The power dissipation from the lesion is also an issue. In particular, the flow of blood through the lesion absorbs much of the energy that otherwise would assist in ablating the tissue. Here, the first treatment may be injection of a substance that reduces, or even better, stops, the flow of blood (a substance such as glue, micro-spheres, micro-coils, a drug for shrinking blood vessels such adrenalin or caffeine, etc.).

The characteristics can be grouped into physical, physiological and biological characteristics. The physical group of characteristics includes: controlling the heat capacity of the tissue, the heat dissipation to and from that tissue and the electrical conductivity to and in that tissue. The physiological group of characteristics includes: changing the vitality of that tissue and controlling the rate of blood flow in the tissue. The biological group of characteristics includes: designating cells by biological markers as targets for later application, implant of receptors for intervening the operation of these cells and drugs for increasing the sensitivity of specific cells to a treatment (such as exposing the cells to toxins) using biological, chemical or genetic engineering.

Examples of substances suitable for use according to the teachings of this patent include but are not limited to: chemicals, bio-chemicals, biological substances, products of genetic engineering materials, micro and nano particles. Additional suitable materials include materials currently used in the cerebrovascular field such as glue, micro-coils and micro-spheres.

Any mechanism for bringing a drug to tissue inside the body can serve for this purpose as part of the first probe. A needle for injection, a sprayer for spraying drug inside the airway, a stent coated by drug and drug delivery mechanism either placed in the airway or implant in the tissue.

One preferred embodiment of the second treatment according to this invention is transferring energy between the second probe and the tissue, for the purpose of ablating a lesion. One common method of ablation is radio frequency (RF) ablation. Serving for RF ablation, the second probe will be a single electrode or set of plurality of ablation electrodes. The electrode (or electrodes) can be a flat electrode or a ring electrode brought in contact with the lesion. In addition, needle electrodes inserted into the lesion can be used. If a single electrode is used, the electric circuit is closed between the electrode and a common electrode attached to the back of the patient. If multiple electrodes are used, the electric circuit can be closed between two or more electrodes.

Another common method for ablation is cryo ablation. In this method, the second probe is a heat pump used for freezing the surrounding of the probe. Yet another method for ablation is radiotherapy. Of particular relevance here are the radioactive seeds for radiating gamma particles. In general, the energy used in context of this patent can be selected from: electrical current, electrical voltage, coherent and non coherent waves of electromagnetic energy includes infrared, visible light, ultra violet, radio frequency, microwaves and gamma radiation, pressure, pressure waves, mechanical shocks, sonic and ultrasonic, emission of particles and thermic energy. In the context of ablation, the first treatment performed by the first probe aimed to increase the effectiveness of ablation caused by the transferred energy, either by increasing the coupling between the probe and the tissue, or by increasing the action of the energy on that tissue, or by increasing the energy insulation between the lesion and the surrounding.

Depending on the required results and the methods of the treatments, the order and the intervals may change from performing both treatments concurrently to performing the second treatments a certain period of time, hours or even days after the first treatment. In addition, the order of the treatments could be reversed by performing the second treatment first and the first treatment after that.

In addition to using energy for the second treatment, otitier methods may also be used. The second treatment may be one of the methods used in the first treatment: application of biochemical, chemical, chemotherapy, biological micro or nano particles, glue or micro coils material into the tissue of the tissue to be treated. In addition, application of drugs can serve for the second treatments such as application of antibiotics, pain relief, anti-inflammatory, gene therapy, enzymatic, bleeding inhibitory and chemo agents materials into the tissue to be treated.

The change of a characteristic of the tissue that is performed by the first treatment may also be done by tissue ablating methods, either by chemotherapy techniques or by using energy in any of the methods described above.

In principle, the location of the first treatment is not necessarily identical to the location of the second treatment even in case the target is a specific lesson. The reason is the principle of operation of each technique. For instance, injecting glue to a lesion may be better done into the veins leading to the lesion, while RF ablation is performed directly on the lesion itself. Hence both locations can be overlapped or displaced from each other, with displacement of translation only, of angular displacement only, or combination of both.

It should be noted that, although illustrated herein in the context of a preferred device for bronchial navigation, the invention in its broader conception may equally be applied using conventional bronchoscopy equipment or other types of endoscopic equipment used for other regions of the body. In each case, a suitable endoscopic localization technique and the corresponding necessary structural components must be employed to ensure correct positioning of the probes for implementation of the treatments.

Reference is also made herein to co-pending, co-assigned U.S. Provisional Patent Application No. 60/564,944 which is herein incorporated by reference. This document, which does not constitute prior art to the present application, teaches additional devices and techniques for localization which may be used to advantage in the context of the present invention.

### EXAMPLE 1

### Local Chemotherapy with Increased Agent Concentration

The metabolism of a cancer lesion is usually high. Injection of agent to such lesion will rapidly dilute the concentration of the agent, as the blood flow carries away a significant portion of that substance. For maintaining the concentration and prolonging its effectiveness, the blood flow should be significantly decreased. In organs such as the arms and legs, it is possible to use a tourniquet to eliminate the blood flow while the chemo agent is affecting the lesion. In the lung, the use of a tourniquet is not possible. Blocking the arteries or reducing blood flow should therefore be done otherwise. In phase one of such a procedure, a substance for reducing the flow rate is injected to the lesion. The substance is injected either into the arteries leading to the lesion in a location proximity to the lesion or into the lesion itself in a location close as possible to the entrance of the arteries. Since the arteries in the lung are clearly seen in the CT data, it is possible to navigate and place the injection needle in the exact location. The substances for eliminating the blood flow can vary from a vaso-constriction drug, such as adrenalin, to total blockage, materials such glue. In phase two, the chemo-agent is injected directly to the lesion.

### EXAMPLE 2

### Reducing the Side Effects of the Local Treatment of Chemotherapy

Application of chemotherapy causes damage to adjacent lung tissue, which may lead to inflammation and infections. Reducing this side effect helps the body to heal better. This is done by application of drugs. The same methods may be used also for the local treatment of chemotherapy done by catheterization. According to the methodology of this patent, in the current example the chemo treatment is the first treatment. The second treatment is injection of drugs for reducing these undesirable side effects. The drug might be one or combination of anti-inflammatory, antibiotic and substance for neutralizing the chemo-agent.

### EXAMPLE 3

### Combining Chemotherapy and Radioactive Seeds

Chemotherapy acts effectively for a short period of time, until it is diluted bellow the effective concentration. Radioactive seeds, on the other hand, may act longer, depending on the isotope type being used. Combining the two for acting locally on the same lesion may increase the probability of completely destroying the lesion. Chemotherapy is applied by injection. Using a locatable guide, a sheath is located for allowing the insertion of tools to the lesion. Through that sheath, a needle is inserted for injecting chemo agent to the lesion. The needle is withdrawn, and a radioactive seed is implanted, either by injection or in the form of a coil placed inside the air path adjacent to the lesion.

### EXAMPLE 4

### Increasing Efficiency of Heat Ablation by Reducing Heat Dissipation

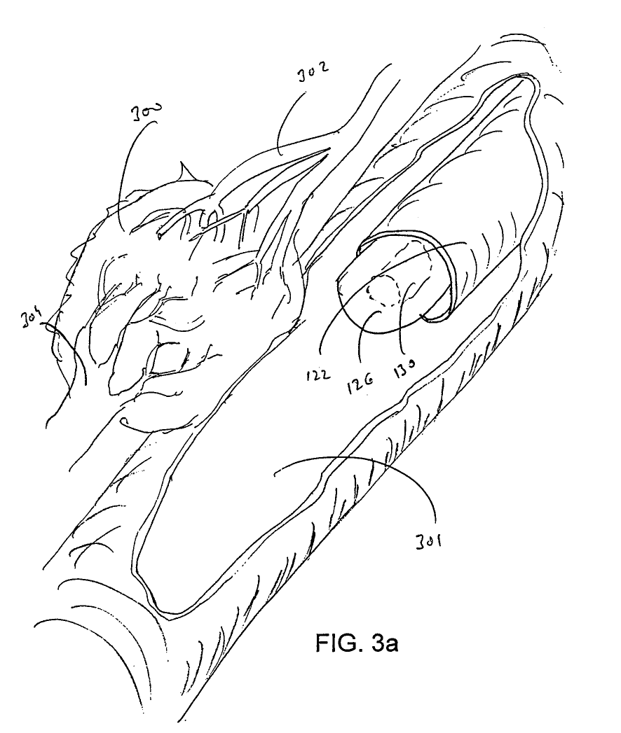

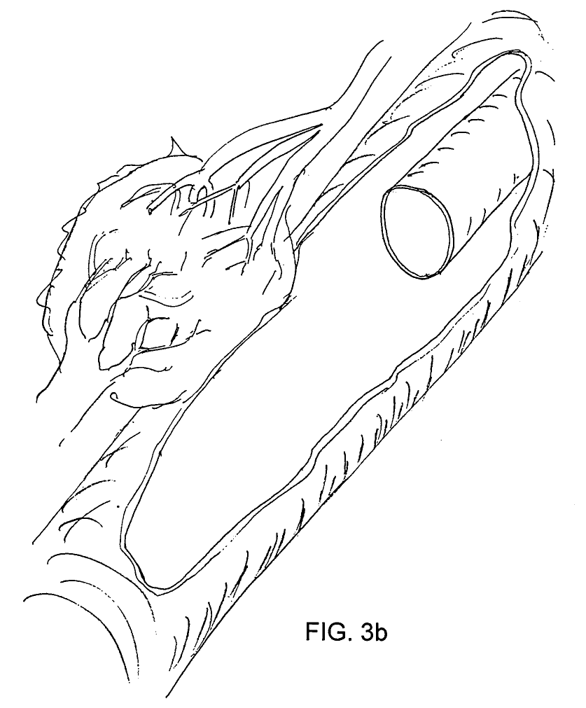

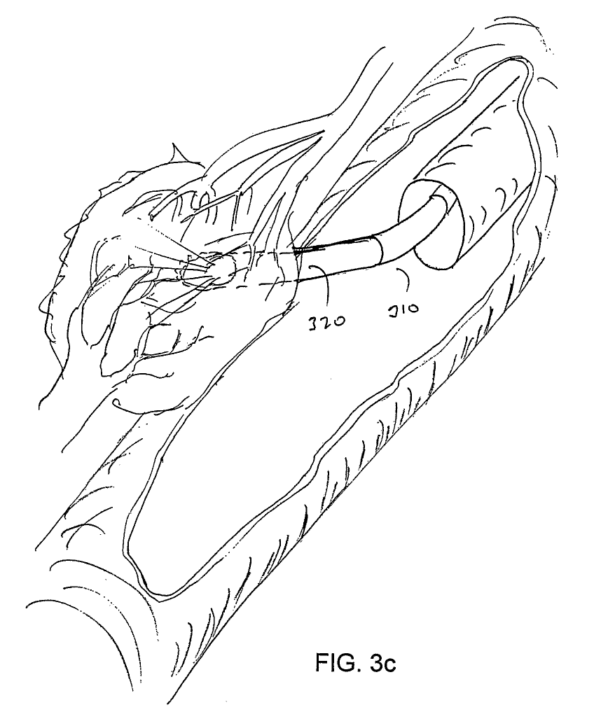

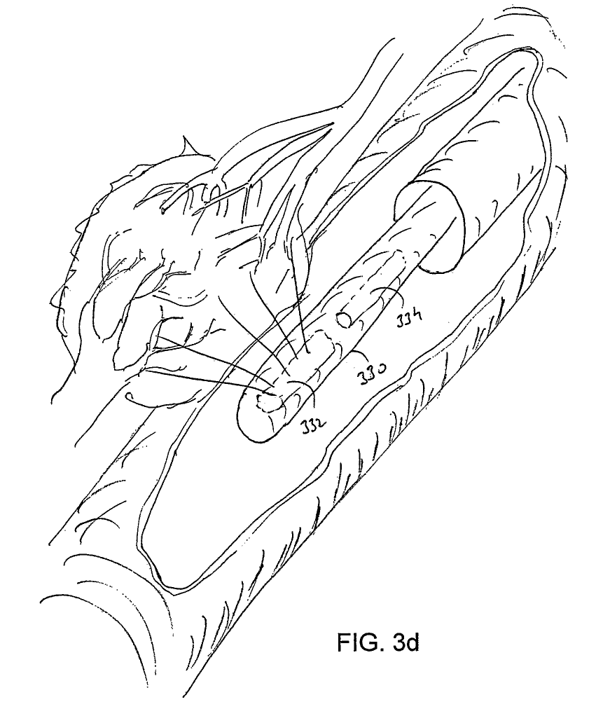

The flow of blood reduces the efficacy of ablation made by heat, since it carries the heat away. By injecting a drug that contracts the blood vessels, the flow is reduced, and in consequence the heat dissipation is also reduced and the efficiency of the ablation process is increased. FIGS. 3a to 3d show the use of the system described in WO 03/086498 in the general method of delivering energy to a lung tissue according to the current concepts. FIG. 3a show an airway 301, where lesion 300 is located adjacent to it. The lesion gets its blood supply from arteries 302, and veins 304 lead the blood out of it. A locatable guide, enveloped with a sheath 122, is navigated inside the body based on the position measurement of the location sensor 130 incorporated at the guide's tip 126. The tip is placed at the lesion and the guide withdrawn, as seen in FIG. 3b. The sheath is used to insert a first catheter 310 having an injection needle 320 at its tip, as shown in FIG. 3c. The needle is directed and pushed into the lesion, and the drug is injected. After the injection is completed, catheter 310 is replaced by a second catheter 330 (FIG. 3d). This catheter aimed to emit energy from energy source 332. The energy is directed to ablate the lesion. Relocation of the tip of the catheter may be needed to fully cover the volume of the lesion. Optionally a location sensor 334 is also incorporate into the tip of the ablation catheter, and repositioning of the catheter's tip may be controlled by the navigation system.

### EXAMPLE 5

### Improved Methods of Ultrasonic Ablation

Another common method to ablate lesions is the use of high-energy ultrasound waves. The efficiency of the process depends on the absorption of acoustic energy in the matter. That depends on the density and the structure of the tissue. Filling the lesion with a matter which is matched to absorb the acoustic energy may assist to ablate the tissue more effectively. The matching element may be micro or nano particles whose mass is resonant at the ultrasound frequency. These particles may be able, in addition, to get absorbed in the body without causing any damage. Such absorbable particles can be made of salt or ice crystals. Other types of absorbable particles can be made from drugs encapsulated in microcapsules. The drugs activated by the mechanical shocks or the heat made by the ultrasound energy. The spreading of the matter in the lesion may be done by injection, by spraying or by gel squeeze into the airway. The ultrasound probe can be a thin internal probe or an external probe. Both are directed by location sensor.

### EXAMPLE 6

### Increasing Absorbing of Laser Energy in a Tissue

Laser energy is a common method for ablating tissue. The wavelength of the light can be selected for low absorption in a living tissue, and good absorption in a special dye matched to the color of the laser. By applying the dye to selected locations, only these locations will be ablated. Application of the dye can be done by injection or spraying. Location sensors can be implemented into the catheter used to apply the dye as well as to the laser probe.

### EXAMPLE 7

### Preventing Migration of Cancerous Cells from a Lesion

The motivation for this procedure is reducing the risk of spreading cancerous cells into the blood and the lymphatic systems during medical intervention. This is done by sealing the lesion before any other procedure performed. The sealant can be a glue or a gel injected to block the veins, or a substance to fill and block the intercellular space. This procedure can be coupled with RF ablation or seed placement.

### EXAMPLE 8

### Targeting a Lesion for a Distance Application

There are treatments to treat a cancer non-invasively from the outside of the body. An example is Radiotherapy. The lesion is irradiated from different directions so to keep the exposure of the healthy tissue at low levels of radiation while the lesion itself receives a high enough dose of radiation to kill the cancerous cells. However, identification of the lesion in the body is critical to the accuracy and the success of this procedure. Injecting contrast agent into the lesion may increase the ability to identify the lesion by a radiotherapy apparatus.

It will be appreciated that the above descriptions are intended only to serve as examples, and that many other embodiments are possible within the scope of the present invention as defined in the appended claims.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for local treatment of a target region of tissue within a body of a subject, the method comprising:
(a) navigating a first probe intra-bodily to a first location within the body of the subject;
(b) employing said first probe to locally change at least one characteristic of a selected region of tissue, said selected region of tissue being in known spatial relation to the target region of tissue; and
(c) employing a treatment probe selected from the group consisting of said first probe and a second probe to apply a treatment to the target region of the body of the subject,
wherein said changed characteristic of said selected region of tissue is chosen so as to enhance at least one parameter of said treatment.

2. The method of claim 1, wherein navigation of said first probe includes measuring a location of said probe relative to the body of the subject.

3. The method of claim 2, wherein said measuring is performed by determining a position of a location sensor associated with said first probe, said location sensor being part of an electromagnetic tracking system.

4. The method of claim 2, wherein said navigation further includes determining a location of said first probe within an image of at least the target region of tissue, said image being derived from a medical imaging system.

5. The method of claim 4, wherein said medical imaging system is selected from the group consisting of: computer tomography, magnetic resonance, nuclear camera, PET and ultrasound imaging.

6. The method of claim 1, wherein said treatment probe is said first probe.

7. The method of claim 6, wherein said first probe includes a first portion configured for changing said characteristic of said selected region of tissue and a second portion configured for applying said treatment.

8. The method of claim 1, wherein said characteristic is a physical characteristic of said tissue.

9. The method of claim 8, wherein said physical characteristic is the heat capacity of said tissue.

10. The method of claim 8, wherein said physical characteristic is the heat dissipation from said tissue.

11. The method of claim 8, wherein said physical characteristic is electrical conductivity of said tissue.

12. The method of claim 1, wherein said characteristic is a physiological characteristic of said tissue.

13. The method of claim 12, wherein said physiological characteristic is the vitality of said tissue.

14. The method of claim 12, wherein said physiological characteristic is the rate of blood flow in said tissue.

15. The method of claim 1, wherein said characteristic is a biological characteristic of said tissue.

16. The method of claim 15, wherein said biological characteristic is changed by marking individual cells.

17. The method of claim 16, wherein said marking said cells is performed by implant of receptors.

18. The method of claim 15, wherein said biological characteristic is changed by increasing the sensitivity of said tissue to the action of a specific material.

19. The method of claim 1, wherein at least one of said first probe and said treatment probe is a needle for injection.

20. The method of claim 1, wherein at least one of said first probe and said treatment probe is a sprayer for spaying a substance to be absorbed into said tissue.

21. The method of claim 1, wherein said first probe includes a stent coated with a drug.

22. The method of claim 1, wherein at least one of said first probe and said treatment probe is a drug delivery device.

23. The method of claim 1, wherein said at least one characteristic of the tissue is changed by application of bio-chemicals into said tissue.

24. The method of claim 1, wherein said at least one characteristic of the tissue is changed by application of chemicals into said tissue.

25. The method of claim 1, wherein said at least one characteristic of the tissue is changed by application of biological materials into said tissue.

26. The method of claim 1, wherein said at least one characteristic of the tissue is changed by application of micro or nano particles into said tissue.

27. The method of claim 1, wherein said at least one characteristic of the tissue is changed by application of glue into said tissue.

28. The method of claim 1, wherein said at least one characteristic of the tissue is changed by application of micro coils into said tissue.

29. The method of claim 1, wherein said treatment includes transferring energy between said treatment probe and the tissue of said target region.

30. The method of claim 1, wherein said treatment is an ablation of tissue in at least part of said target region and wherein said treatment probe is an ablation device.

31. The method of claim 29, wherein said transferred energy is radiofrequency ablation energy and said treatment probe is an electrode for delivering said radiofrequency ablation energy.

32. The method of claim 29, wherein said treatment probe is a heat absorber for freezing said tissue.

33. The method of claim 29, wherein said treatment probe includes a radioactive emitter and said transferred energy is radioactive radiation.

34. The method of claim 33, wherein said radioactive radiation is gamma radiation.

35. The method of claim 29, wherein said energy is selected from the group consisting of: electrical energy, coherent and non-coherent electromagnetic waves including infrared, visible light, ultra violet, radio frequency, microwaves and gamma radiation, pressure, pressure waves, mechanical shocks, sonic, ultrasonic, emission of particles and thermal energy.

36. The method of claim 29, wherein said changing is effective to increase an energy coupling between said treatment probe and the tissue of said target region.

37. The method of claim 29, wherein said changing is effective to increase an energy insulation between the tissue at said target region and at: least one adjacent region of tissue.

38. The method of claim 30, wherein said changing is effective to increase a number of dead cells in the tissue at said target region.

39. The method of claim 1, wherein said treatment is applied while said treatment probe is positioned substantially at said first location.

40. The method of claim 1, wherein said treatment is applied while said treatment probe is positioned at a second location displaced relative to said first location.

41. The method of claim 1, wherein said treatment includes application of bio-chemicals into said tissue.

42. The method of claim 1, wherein said treatment includes application of chemicals into said tissue.

43. The method of claim 1, wherein said treatment is chemotherapy.

44. The method of claim 1, wherein said treatment includes application of biological materials into said tissue.

45. The method of claim 1, wherein said treatment includes application of micro or nano particles into said tissue.

46. The method of claim 1, wherein said treatment includes application of glue into said tissue.

47. The method of claim 1, wherein said treatment includes application of micro coils into said tissue.

48. The method of claim 1, wherein said changing at least one characteristic of said tissue is effected from ablation of said tissue.

49. The method of claim 48, wherein said ablation of tissue is performed by chemotherapy techniques.

50. The method of claim 48, wherein said ablation of tissue is performed by an energy method of ablation.

51. The method of claim 48, wherein said treatment includes application of a drug at said target region.

52. The method of claim 51, wherein said drug is selected from the group consisting of: antibiotics, pain relief, anti-inflammatory, gene therapy, enzymatic drug, bleeding inhibitory and chemo-agents.

53. The method of claim 1, wherein said changing said at least one characteristic of said tissue and said treatment are performed concurrently.

54. The method of claim 1, wherein said changing said at least one characteristic of said tissue is performed after said treatment.

55. The method of claim 1, wherein said navigating is performed using at least one endoscopic localization technique selected from the group consisting of: electromagnetic location sensing; magnetic location sensing; image correlation; and trigonometry based upon at least one non-invasive imaging device.

56. The method of claim 1, wherein said selected region of tissue is substantially coincident with said target region of tissue.

57. The method of claim 1, wherein said selected region of tissue is substantially adjacent to said target region of tissue.
